(19) United States
(12) Patent Application Publication
(10) Pub. No.: US 2004/0005304 A1
(43) Pub. Date: Jan. 8, 2004

(54) Novel compositions and methods for treating neurological disorders and associated gastrointestinal conditions

(75) Inventor: Mark A. Brudnak, Port Washington, WI (US)

Correspondence Address: Sara D. Vinarov, Michael Best & Friedrich LLP, One South Pinckney Street, P.O. Box 1806, Madison, WI, 53701-1806, US

(73) Assignee: MAK Wood, Inc., Grafton, WI

(21) Appl. No.: 10/191,385

(22) Filed: Jul. 8, 2002

(51) Int. Cl. A61K 45/00; A61K 38/16; A61K 38/10; A61K 38/08; A61K 38/06; A61K 38/04; A61K 31/70

(52) U.S. Cl. 424/93.45; 514/2; 514/12; 514/13; 514/14; 514/15; 514/16; 514/17; 514/18; 514/19; 514/23

(57) ABSTRACT

The present invention provides therapeutic compositions and methods for treating to neurological disorders and associated gastrointestinal conditions using enhancer molecules. These enhancer molecules comprise therapeutically effective amounts of metals, amino acids, polypeptides, saccharides, probiotics, and combinations thereof to enhance expression of genes, and/or enzymatic activity of gastrointestinal proteins.

### STATEMENT REGARDING FEDERALLY FUNDED RESEARCH

Not Applicable.

### STATEMENT REGARDING CROSS REFERENCES

Not Applicable.

### TECHNICAL FIELD

The present invention relates to a composition and method for treating neurological disorders and associated gastrointestinal conditions.

### BACKGROUND OF THE INVENTION

Currently, there are several prevailing therapies that attempt to address general neurological diseases, and associated gastrointestinal conditions: (1) diet restriction; (2) surgical treatment; (3) steroid administration; (4) diet supplementation with exogenous enzymes; and (5) diet supplementation with probiotics. Thus far, none of these therapies individually, or in combination, have been found to be totally effective in the treatment of neurological pathologies and associated gastric problems. With respect to diet restriction, typically, casein free products and gluten free products are suggested. However, restricting the diets of young children is difficult, particularly in school or caregiver settings. For example, autism diets typically restrict gluten, which restrict the child from eating most bread products, and limit most convenient meal options. With respect to enzyme therapy, treatment usually consists of acid/carboxyl peptidase, endo peptidase, or zinc protease diet supplements in pill or powder form. This treatment does not provide an effective solution either, mainly because these added enzymes may perform their function in the stomach before intestinal absorption can occur.

Furthermore, with respect to probiotic supplementation, this treatment mainly functions to rebuild the patient's intestinal walls. In general, probiotics are any live bacterial culture taken orally to improve the health of the gastrointestinal tract. If the gastrointestinal tract is not functioning properly, as is the case in diseases such as autism, the probiotic supplements will theoretically begin to restore the bacterial balance in the intestines. Generally the bacterium, Lactobacillus acidophilus is used, but wide ranges of bacterial varieties are available to be used as probiotics. Also, there are compounds called “pre-biotics,” which are used to nourish the probiotics. The theory behind this therapy is that by adding pre-biotics, the growth of probiotics can be stimulated, thereby increasing the bacterial flora and decreasing the symptoms related to the autistic dysbiosis. However, the probiotic therapy has not been shown to act fast enough, or to the extent necessary to counteract the uptake of the opioids that exist through the gut walls or that may be present in the blood at time of administration of the probiotics.

However, while probiotic supplementation appears to provide a promising treatment, results are currently inconsistent. For example, adding foreign bacteria to the gut can be somewhat problematic, particularly when the bacteria is from an isolated ‘pure’ strain, because the environment of the gastrointestinal tract may result in the death of certain bacteria, and those that survive may have minimal efficacy. Moreover, the difficulty associated with isolating bacteria, growing a culture, and shipping the live product to the consumer across a large temperature range and variety of conditions without significant loss of bacteria or risk of contamination is quite high. Therefore, a method of increasing the efficacy of desired bacteria, contained in the probiotic, remains desired.

Current theories indicate that diets play an important role in the development of neurological disease and associated gastrointestinal conditions. Neurological disease, particularly autism, is a developmental condition affecting as many as 1 in 300 children. The condition is often characterized as a mental disorder originating in infancy that is associated with self-absorption, the inability to interact socially, behavior, and language dysfunction. It is thought that, as a result of maldigestion of casein and gluten, opioid type peptides are produced. Research has suggested the presence of peptides with opioid properties in the urine of autistic subjects is consistent with the hypothesis that the syndrome could be the result of the brain's uptake of neuroactive diet-derived opioid compounds from the gut, suggesting that an abnormal gut permeability or “leaky gut” produces the disorder (Reichelt et al. 1991, Brain Dysfunct., 4:308-319; Reichelt et al. 1994, Dev. Brain Dysfunct., 7:71-85; both of which are specifically incorporated by reference, herein).

In autistic patients, the body's intestinal epithelial lymphocytes ‘overbind’ to mycobacterial antigens present in the gut, and effectively the body ‘downregulates’ protease genes, such as the DPPIV gene. When the body stops making enough of these protease genes, it cannot effectively break down proteins in the diet. Normally, these proteins would not be able to pass from the gut lumen into the blood between epithelial cells due to the presence of tight junctions between the cells. However, in autistic patients a “leaky gut” occurs wherein inflammation causes the disruption of these tight junctions, resulting in increased uptake of further toxins or inflammatents, thus repeating the cycle. The direct link between autism and “leaky gut” is not known, however, autism is commonly associated with gastrointestinal gut disorders.

Currently, no therapy effectively addresses the molecular mechanism that may be at work in the development and progression of neurological conditions. Therefore, as an alternative therapy, the present invention provides compositions and methods for treating neurological disorders and associated gastrointestinal conditions at the structural genomics level and the functional proteomics level.

### SUMMARY OF THE INVENTION

The present invention provides therapeutic compositions and methods for enhancing gene expression and/or the activity of enzymes that are present in the gastrointestinal tract, and wherein a drop in their activity has been linked to neurological disorders.

The present invention provides compositions for the treatment of neurological disorders and associated gastrointestinal conditions. The compositions may comprise a therapeutically effective amount of at least one of a peptide, a saccharide, a probiotic, a metal and combinations thereof. The compositions may have the ability to enhance expression of genes, enhance enzymatic activity of gastrointestinal proteins, or both.

The present invention further provides a method for treating neurological disorders and associated gastrointestinal conditions. The method may comprise administering therapeutically effective amounts of a composition which may have at least one of a peptide, a saccharide, a probiotic, a metal and a combination thereof. The method may have the ability to enhance expression of genes, enhance enzymatic activity of gastrointestinal proteins, or both.

Particularly, this invention may relate to compositions and methods for treatment of one or more of the following non-limiting list of neurological conditions such as: autism and autism spectrum disorders; Parkinson's disease; cognitive impairments; age-associated memory impairments; cognitive impairments; dementia associated with neurologic and/or psychiatric conditions; epilepsy; brain tumors; brain lesions; multiple sclerosis; Down's syndrome; Rett's syndrome; progressive supranuclear palsy; frontal lobe syndrome; schizophrenia and related psychiatric disorders; delirium; Tourette's syndrome; myasthenia gravis; attention deficit hyperactivity disorder; dyslexia; mania; depression; apathy; myopathy; Alzheimer's disease; Huntington's Disease; dementia; schizophrenia; severe clinical depression; brain injury; ADD (Attention Deficit Disorder); ADHD (Attention Deficit Hyperactivity Disorder); hyperactivity disorder; Asperger's Disorder; and other pervasive developmental disorders.

The invention may also relate to compositions and methods for the treatment of one or more of the following non-limiting list of gastrointestinal conditions commonly associated with neurological diseases such as: nausea, abdominal pain, constipation, diarrhea, Functional Bowel Disorder, Irritable Bowel Syndrome, Crohn's disease, ulcers, heartburn, irregularity, gastric neurosis, diverticulosis, cirrhosis, celiac disease, acute gastritis, dyspepsia, gastralgia, gastric carcinoma, gastric vertigo, enteritis, peptic ulcers, cholera morbus, cholera infantum, gastroenteritis, bloating, flatulence, inflammatory bowel disease, acid reflux disease, abdominal pain, and Ulcerative colitis.

The patents, references and articles cited herein are hereby fully incorporated by reference. In the case of conflict between the present disclosure and the incorporated patents, references and articles, the present disclosure should control.

### BRIEF DESCRIPTION OF THE DRAWINGS

Not applicable.

### DETAILED DESCRIPTION OF THE INVENTION

The present invention relates broadly to ameliorating or preventing neurological disorders and associated gastrointestinal conditions. Accordingly, the present invention will now be described in detail with respect to such endeavors; however, those skilled in the art will appreciate that such a description of the invention is meant to be exemplary only and should not be viewed as limitative on the full scope thereof.

More specifically, the present invention relates to therapeutic compositions and methods for enhancing or increasing the expression of genes and/or the activity of enzymes, in the gastrointestinal tract, wherein a reduction in activity has been linked to neurological disorders.

The present invention provides for a composition, the formulation of which may comprise a combination of a metal enhancer, a peptide or a peptide derivative (ranging in length from 1-30 amino acids), a mono-, poly-saccharide, or a salt thereof together with one or more carriers or excipients, and gastrointestinal probiotic analogues wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a mono- di-, tri-peptide, a polypeptide (ranging in length from 1 to 30 amino acids) or a derivative thereof together with one or more carriers or excipients, wherein the amino acids or peptides are capable of entering the intercellular fluid and is present in an amount sufficient to therapeutically enhance expression of genes or enhance enzymatic activity of gastrointestinal tract enzymes, resulting in an amelioration or prevention of neurological disorders with associated gastrointestinal conditions.

The present invention provides for a composition, the formulation of which may comprise a metal enhancer together with one or more carriers or excipients, wherein the metal enhancer is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a mono-, poly-saccharide, a salt thereof, or a combination thereof together with one or more carriers or excipients, wherein the polyglycoside is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a at least one probiotic organism containing an analogue of a gastrointestinal enzyme, as defined herein, an example of which is an analogue of the DPPIV enzyme that is known to be able to digest exorphins, wherein the probiotic analogues are capable of entering the intercellular fluid and are present in an amount sufficient to therapeutically enhance expression of genes or enhance the activity of gastrointestinal tract enzymes, so as to ameliorate or prevent, neurological disorders with associated gastrointestinal conditions.

The present invention provides for a composition, the formulation of which may comprise a variety of probiotic organisms that may be found to contain a collection of genes which code for a variety of proteins (i.e. probiotic analogues) that exhibit enzyme activity capable of breaking-down exorphins, wherein the probiotic analogues are capable of entering the intercellular fluid and are present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a metal enhancer, and a peptide or a peptide derivative (ranging in length from 1-30 amino acids) together with one or more carriers or excipients, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a metal enhancer, a peptide or a peptide derivative (ranging in length from 1-30 amino acids) together with one or more carriers or excipients, and gastrointestinal probiotic analogues wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, formulation of which may comprise a combination of a metal enhancer, a mono-, poly-saccharide, or a salt thereof together with one or more carriers or excipients, and gastrointestinal probiotic analogues, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a metal enhancer, a mono-, poly-saccharide, or a salt thereof together with one or more carriers or excipients, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a metal enhancer, together with one or more carriers or excipients and gastrointestinal probiotic analogues, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, formulation of which may comprise a combination of a mono-, poly-saccharide, or a salt thereof together with one or more carriers or excipients and gastrointestinal probiotic analogues, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a peptide or a peptide derivative (ranging in length from 1-30 amino acids) together with one or more carriers or excipients, and gastrointestinal probiotic analogues wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, formulation of which may comprise a combination of a metal enhancer, and a mono-, poly-saccharide, or a salt thereof together with one or more carriers or excipients, and a peptide or a peptide derivative ranging in length from 1-30 amino acids, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

The present invention provides for a composition, the formulation of which may comprise a combination of a peptide or a peptide derivative (ranging in length from 1-30 amino acids) and a mono-, poly-saccharide, or a salt thereof together with one or more carriers or excipients, wherein the composition is capable of entering the intercellular fluid and is present in an amount sufficient to therapeutically enhance expression of genes or enhance the activity of gastrointestinal tract enzymes, resulting in an amelioration or prevention of neurological disorders with associated gastrointestinal conditions.

Definitions

Unless otherwise noted, technical terms are used according to conventional usage. In order to facilitate review of the various aspects of the invention, the following definitions of terms are provided:

The term “enzyme enhancer” or “enhancer molecule” as referred to herein may include any or all molecules that would positively affect the activity of an enzyme as defined by the conversion of a unit of substrate per unit of time. Furthermore, an enzyme enhancer may increase an enzyme's normal function, beyond the increase observed with an enzymatic co-factor. For example, enhancer molecules allow the enzyme to perform the same function as a co-factor, but for a longer period of time. The net result is that the same amount of enzyme produces a greater amount of activity.

More particularly, an enhancer molecule can be naturally occurring or synthetic compounds that when ingested can cause a gene to either alter its expression pattern (i.e., make more or less of the product), affect the fidelity of a gene (i.e., how well that gene product works) or affect the integrity of a gene (i.e., whether or not the gene is functional). Enzyme enhancers do not directly replace substances that are missing (e.g., an enzyme activity diminished by genetic mutation), but actually alter the expression and/or functionality of gene products resulting from the genetic mutation. Therefore, enzyme enhancers have the ability to increase gene expression by affecting genetic elements that may be acting at any given loci (promoter, enhancers, transcription factors, etc.), and to also affect the RNA transcript, and/or the translated protein exhibiting enzymatic activity.

As described in following sections, the term “enzyme enhancer” may represent peptides (ranging in length from 1-30 amino acids), saccharides (ranging in length from 1-15 carbohydrates), probiotic organisms (containing genes encoding a protein that exhibits enzyme activity capable of breaking-down exorphins), and metals or combinations thereof.

Dipeptides such as (Gly-L-Ile) are an example of such enzyme enhancers that have been shown to increase the level of transcription and translation of important genes. Also adding dipeptides and other peptides ranging in length from 1 to 10 amino acids to a diet has been shown to increase both RNA and protein levels. Moreover, the addition of dipeptides also increases the expression of Dipeptidyl peptidase IV (DPPIV), again suggesting an expression-based response to the presence of dipeptides.

It is understood that enzyme enhancers can be used to ameliorate or prevent many human neurological disorders and their associated gastrointestinal conditions such as, for example: ASD, lactose maldigestion, celiac disease (and other similar gastrointestinal disorders), mitochondrial diseases, Alzheimer's disease (enzymes involved in the processing of amyloid beta-peptide), age-related diseases, cystic fibrosis, Thyroid disease, cancer, familial amyotrophic lateral Sclerosis, superoxide dismutase (SOD) related disorders, and other related diseases that have yet to be identified. These are examples of diseases where enzyme levels have been found to be attenuated, as compare to wild-type (normal) enzyme levels. It is also known that such enhancers may function by altering the conformation (shape) of the three-dimensional enzyme structure in such a way so as to increase the affinity of the active site on the enzyme for the substrate (target molecule). This may be accomplished either by directly altering the enzymatic active site or some other portion of the enzyme (i.e., metal binding motif, cofactor binding region, substrate binding site, etc. . . . ), thus creating a novel structure which is better able to process the substrate.

The term Dipeptidyl peptidase IV or “DPPIV” as referred to herein may include any or all DPPIV probiotics, including analogues of human DPPIV genes and any functional analogues thereof. DPPIV is a serine exoproteinase expressed at high levels in epithelial cells of kidney, liver and small intestine, and functions, in part, as a protease, or enzyme that can break down other proteins (Thompson et al. 1991, Biochem. J. 273:497-502).

DPPIV is widely produced in mammalian tissues and fluids and functions to cleave Xaa-Pro dipeptides from the N-terminus of oligo- and polypeptides. DPPIV is produced in sufficient quantities naturally by the body, however autistic patients produce a reduced amount of the normal DPPIV required by the body. The treatments for autism described herein, depend on increasing the normal production of DPPIV, increasing the amount of DPPIV in the gut, and improving the efficacy of the DPPIV already present in the gut.

Furthermore, DPPIV is unusual because it can be activated by “enhancer molecules” or “enzyme enhancers” such as individual amino acids and peptides. (See Shibuya-Saruta et al. 1996, J. Clin. Lab. Anal. 10:435-440, which is specifically incorporated by reference, herein). Shibuya-Saruta et al. demonstrate that the level of DPPIV is increased in the gastrointestinal tract resulting in an increase in overall human digestion, which may assist the body in breaking down neuroactive diet-derived opioid compounds before they travel to the brain.

The term “Prolinase gene” or (pepR) as referred to herein may include any or all Prolinase-containing probiotics, including analogues of Prolinase genes and any functional analogues thereof. Prolinase gene encodes for a protein that exhibits enzyme activity capable of breaking-down exorphins. It is contained in a variety of probiotic organisms. (See Varmanen P, Rantanen T, Palva A, Tynkkynen S. Cloning and characterization of a prolinase gene (pepR) from Lactobacillus rhamnosus, Appl. Environ. Microbiol. 1998 May; 64(5):1831-6).

The term “proline iminopeptidase gene” or (pepI) as referred to herein may include any or all proline iminopeptidase-containing probiotics, including analogues of proline iminopeptidase genes and any functional analogues thereof. Proline iminopeptidase gene encodes for a protein that exhibits enzyme activity capable of breaking-down exorphins. It is contained in a variety of probiotic organisms. (See Varmanen P, Rantanen T, Palva A. An operon from Lactobacillus helveticus composed of a proline iminopeptidase gene (pepI) and two genes coding for putative members of the ABC transporter family of proteins, Microbiology. 1996 Dec;142 (Pt 12):3459-68).

The term “gastrointestinal tract enzymes,” as referred to herein, may include all hydrolases, such as glycanases (i.e., all carbohydrate-cleaving enzymes, including endo- and exo-glycosidases), synthethases (i.e., all carbohydrate-adding enzymes, such as glycosyl- and galactosyl-transferases), proteases (i.e., all polypeptide cleaving enzymes), lipases (i.e., fat- or lipid-cleaving enzymes) oxidases and reductases.

The term “gene expression” as referred to herein, refers to the expression from the genes via genetic elements that may be acting at any given chromosomal loci (i.e. promoter, enhancer, transcription factor, etc. . . . ) to affect proper transcription and translation of the gene product.

The term “metal ions or metal enhancer” as referred to herein, refers to ions that can act as catalysts for enzymatic reactions to increase activity by possibly acting as a redox reagent in an enzymatic reaction. Likewise, in the instant invention, metal ions act as natural co-factors, and may positively increase the activity of gastrointestinal enzymes, such as DPPIV, by improving the ability of DPPIV or DPPIV-like enzymes to cleave proline-containing peptide bonds in exorphins. Examples of metals which can activate enzymes include, but are not limited to the following: iron, iron-sulfur, copper, manganese, selenium zinc and aluminum. These enhancer metals, alone, or in combination with amino acids, peptides or sugars, could be utilized in the present invention as a means of stimulating enzymes in the gastrointestinal tract.

The terms “synergy”, “synergism” or “synergistic” referred to herein, refers to a phenomena, where the effect of the two chemicals acting together is greater than the simple sum of their effects when acting alone. As it relates to the present invention, the term synergy refers to the multiplied affect that is achieved from combining any two or all of a metal enhancer; a peptide or a peptide derivative (ranging in length from 1-30 amino acids); and a mono-, poly-saccharide, or a salt thereof together with one or more carriers or excipients and gastrointestinal probiotic analogues, so that when the formulation enters the intercellular fluid, it can more effectively and therapeutically enhance the abnormally low activity of gastrointestinal tract enzymes and assist the body in breaking down neuroactive diet-derived opioid compounds before entry into the brain, resulting in neurological disorders with associated gastrointestinal conditions.

The term “probiotic” dietary supplement as used herein, may refer to bacteria (Lactobacillus bulgaricus, Lactobacillus acidophilus, Lactobacillus plantarum, and Bifidobacterium bifidum) which favorably alter the intestinal micro-flora balance, inhibit the growth of harmful bacteria, promote good digestion and intestinal health, boost immune function, and increase resistance to infection in individuals. Individuals with flourishing intestinal colonies of beneficial bacteria are better equipped to fight the growth of disease-causing bacteria. Probiotics are important in finishing the digestive process and therefore can increase the absorption of nutrients.

Compositions for administration of the active agent in the method of the invention may be prepared by means known in the art for the preparation of compositions (such as in the art of veterinary and pharmaceutical compositions) including blending, grinding, homogenising, suspending, dissolving, emulsifying, dispersing and where appropriate, mixing of the active agent, together with selected excipients, diluents, carriers and adjuvants.

As indicated hereinabove, probiotic dietary supplementation has focused mainly on trying to rebuild the intestinal wall via a restoration of the naturally occurring bacterial flora. In general, common intestinal microflora are isolated from the human gut and cultured to form a “probiotic culture.” Two approaches for the treatment of neurological disorders (such as autistic dysbiosis) and their associated gastrointestinal conditions have emerged, using probiotic cultures. The first approach uses a wide variety of micro-organisms in an effort to attempt restoration of the naturally occurring bacterial flora and is something of a “shot-gun” approach. The second employs a more targeted approach by supplying a “high dose” of viable organisms such as multiple billions of micro-organisms per gram of culture. Both approaches have been based on clinically observed and published therapeutic benefits of probiotics and while they have met with limited success relative to no probiotic treatment, they do not adequately treat the syndromes associated with autism, suggesting that exorphin digestion is more than just an issue of microflora composition.

In addition, galactose serves as a fuel source of the beneficial microflora (i.e., probiotics) in the gut. This is important because the probiotic organisms themselves contain enzymes capable of breaking-down the subject exorphins. (See Varmanen P, Savijoki K, Avall S, Palva A, Tynkkynen S. X-prolyl dipeptidyl aminopeptidase gene (pepX) is part of the glnRA operon in Lactobacillus rhamnosus, J Bacteriol. 2000 Jan;182(1):146-54, which is specifically incorporated by reference, herein.) Varmanen et al., recently demonstrated that probiotic organisms (i.e., L. rhamnosus), currently utilized as health supplements, contain an analogue of the DPPIV enzyme (e.g., PepX) which is known to be able to digest exorphins. Independent assays have also been conducted (data not presented herein) demonstrating that other strains (i.e., L. helveticus) exhibit DPPIV activity at a much higher rate. With over 1×10¹¹ microorganisms in the gut, the contribution of probiotic enzymatic activity may exceed that of the enterocytes. Accordingly, it is well documented that galactose is a pre-biotic (i.e., stimulates growth of probiotics) and can increase the number of probiotics in the gut.

Furthermore, probiotic organisms may be found to contain a collection of genes which code for a variety of proteins that exhibit enzyme activity capable of breaking-down exorphins. Examples of these genes that exhibit enzyme activity capable of breaking-down exorphins include but are not limited to prolinase gene (pepR) and proline iminopeptidase gene (pepI). (See Varmanen P, Rantanen T, Palva A, Tynkkynen S. Cloning and characterization of a prolinase gene (pepR) from Lactobacillus rhamnosus, Appl. Environ. Microbiol. 1998 May; 64(5):1831-6; and Varmanen P, Rantanen T, Palva A. An operon from Lactobacillus helveticus composed of a proline iminopeptidase gene (pepI) and two genes coding for putative members of the ABC transporter family of proteins, Microbiology. 1996 Dec;142 (Pt 12):3459-68; each of which is specifically incorporated by reference, herein).

The term “amino acid” referred to herein, refers to naturally occurring amino acids, as well as synthetic analogs and derivatives thereof. In particular, it refers to the amino acids that have been found to activate and increase gastrointestinal enzymes such as DPPIV. These amino acids include Alanine, Arginine, Glycine, Glutamine, Glutamic Acid, Isoleucine, Phenylalanine, Serine, Threonine, and Valine.

Furthermore, the term “amino acid” or “amino acid residue” includes conservative derivatives of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups.

In general the abbreviations used herein for designating the amino acids and the protective groups are based on recommendations of the IUPAC-IUB Commission on Biochemical Nomenclature (Biochemistry 1972, 11:1726-1732, which is specifically incorporated by reference, herein). These amino acid residues are abbreviated as follows: Alanine is Ala or A, Arginine is Arg or R, Glycine is Gly or G, Glutamine is Gin or Q, Glutamic Acid is Glu or E, Isoleucine is Ile or I Phenylalanine is Phe or F; Serine is Ser or S, Threonine is Thr or T, and Valine is Val or V. The symbol X may be used to refer to any one amino acid of the preceding group, or any other naturally occurring amino acid.

The term “dipeptide” referred to herein, refers to the linkage of two naturally occurring amino acids or synthetic analogs or conservative derivatives together to form a dipeptide. Dipeptides primarily include, but are not limited to one or more selected from the group consisting of the following: Gly-Ala, Gly-Gly, Gly-Gly-Gly, Gly-Ile, Gly-D-Ala, Gly-Arg, Gly-Asp, Gly-Asn, Gly-Gln, Gly-Glu, Gly-Leu, Gly-Phe, Gly-Pro, Gly-Ser, and Gly-Thr.

The term “Exorphin” referred to herein, refers to external substances that have opiate-like (narcotic) activity in the body, thereby acting like the body's own narcotics, the endorphins. The two primary examples of exorphins relevant to the present application are casomorphins and gluteomorphins. By providing enzymatic enhancers as in the present invention the production of exorphins may be inhibited. The opioid bioactivity of exorphins is reduced, by action of an exorphinase, which alters the structure or composition of the exorphins, for example by oxidation, reduction or cleavage, or by action of an exorphin ligand, which binds to the exorphin. For example, “casomorphin” is an exorphin derived from casein (i.e., milk products) via the activity of proteases, possibly including chymotrypsin and trypsin, as well as indirectly by the action of gastric hormones such as secretin. Also, for example, “gluteomorphin” is an exorphin derived from gluten (i.e., wheat products) via the activity of proteases, possibly including chymotrypsin and trypsin, as well as indirectly by the action of gastric hormones such as secretin.

The term “sugar” or “monosaccharide” as referred to herein may include, but is not limited to the following: simple sugars including pentose, ribose, hexose, glucose, fructose, mannose, glucosamine, galactose, galactosamine, xylose and monoglycoside sugar analogs or conservative derivatives thereof.

The term “polysaccharide” as referred to herein may include more complex sugars such as lactose, sucrose, iduronic acid, glycimic acid, starches, dextrin oligoglycosides and sugar analogs.

Where the polysaccharide chain is substituted regularly, it may comprise a repeating glycosidic unit. Preferably the repeating glycosidic unit is a mono-, di-, tri- or polysaccharide unit.

The term “salt” as referred to herein may include, but is not limited to calcium, sodium, magnesium, potassium or ammonium salt.

The anionic substituent may be selected from the group consisting of sulphate (e.g., SO₃⁻or HSO₃⁻) and carboxylate (e.g., CO₂⁻or HCO₂). In a preferred embodiment, the anionic substituent is sulphate. In this case, there may be one, two or three sulphate substituents per glycosidic residue.

The anionic substituent may be directly or indirectly ring-bound. For example, the anionic substituent may be bound to the ring by a bridging group such as for example an epoxy group, an optionally alkyl-substituted imino group or an alkoxo (e.g., CH₂O) group.

The polysaccharide may be a natural or synthetic polyglycoside and may be homogeneous or heterogeneous. The glycosidic chain may be linear or non-linear. Typically the molecular weight of the polyglycoside is in the range 1 kDa to 8 MDa.

These mono- and poly-saccharides are able to pass from the gut lumen into the blood by traversing epithelial cells but cannot normally pass between cells due to the presence of tight junctions. Toxins and inflammation are two potential causes of disruption of tight junctions. Once disrupted, the effect will tend to be potentiated by the enhanced uptake of further toxins and/or the inflammatory response resulting from the initial breach of the epithelial barrier.

In general, sugars or saccharides function in two ways to treat neurological disorders, such as autism. Sugars can increase the activity of the gastrointestinal enzymes, such as DPPIV. Sugars can also serve as a fuel source for other beneficial microflora in the gut, which in turn digest exorphins. To counteract a “leaky gut”, sugars or complex molecules containing sugars alone may be given to patients to “tighten” the junctions found between gut epithelial cells. Compounds like galactose may reverse or circumvent the down-regulation of the DPPIV gene, tightening the junctions in the intestinal walls. Smith et al. have reported that galactose can increase the expression of DPPIV in cultured mouse intestinal wall cells (also known as enterocyte cells). (see Smith, M. W., James, P. S., Peacock, M. A., Galactose Effects on Enterocyte Differentiation in the Mouse Jejunum. Biochem. Biophys. Acta Jul. 10, 1991, 1093(2-3):1446).

Furthermore, with respect to gastrointestinal disorders, without wishing to be bound by any theoretical considerations, it is understood that preventing or reducing inflammation in accordance with the invention leads to an improvement of the tight junctions found between gut epithelial cells which thereby inhibits the uptake of neuroactive compounds that may be causing the psychological symptoms of neurological disorders, such as autism. The polyanionic polysaccharides are thought to interact with cellular heparin binding sites which are found on proteins on the cell surface. As these proteins are taken into the cell to be either destroyed or recycled, any compound bound to the heparin binding site enters the cell as well and has an effect therein. For instance, specific cytotoxic cytokinins may cause apoptosis in epithelial cells and lead to severe ulceration and further inflammation of the gut surface. Polyanionic polyglycosides present in the intercellular fluid also bind with heparin binding sites (and some would displace the normal compounds such as heparin) on cytokinins and when they themselves are taken into the cells, they are destroyed with the cytokinin and no effect by these compounds is seen. As such, the presence of polyanionic polysaccharides may prevent inflammation and damage caused by cellular chemicals.

As used herein, the term “gastrointestinal conditions or disorder or disease” is intended to cover all gastrointestinal conditions affected, exacerbated, or caused by the body's inadequate breakdown of proteins and peptides. Examples of such gastrointestinal conditions encompassed within the scope of the invention, may be: nausea, abdominal pain, constipation, diarrhea, Functional Bowel Disorder, Irritable Bowel Syndrome, Crohn's disease, ulcers, heartburn, irregularity, gastric neurosis, diverticulosis, cirrhosis, celiac disease, acute gastritis, dyspepsia, gastralgia, gastric carcinoma, gastric vertigo, enteritis, peptic ulcers, cholera morbus, cholera infantum, gastroenteritis, bloating, flatulence, inflammatory bowel disease, acid reflux disease, abdominal pain, and Ulcerative colitis.

As used herein, the term “Autism” refers to a childhood psychosis originating in infancy which is characterized by a wide spectrum of psychological symptoms that progress with age (e.g., lack of responsiveness in social relationships, language abnormality and a need for constant environmental input). It generally appears in children between the ages of two and three years and gives rise to a loss of the development previously gained by the child. The syndrome frequently leads to repeated narrow spectrum diets and psychological difficulties in changing other aspects of life. Epilepsy commonly develops after the age of ten and many drugs are used to control this. The child may develop into an adult that can not be involved normally in society or generate its own income.

Autism is commonly associated with certain abdominal complaints (such as abdominal pain, nausea, retching, constipation, inflammatory bowel disease and malabsorption). The abdominal complaints have been looked upon for some time as being separate to the psychological symptoms or caused by them (Wing, 1997, Autism, 1:13-23; which is specifically incorporated by reference, herein) and have therefore undergone little investigation.

As used herein, the term “neurological disorder” is intended to cover all neurological conditions affected, exacerbated, or caused by the body's inadequate breakdown of proteins and peptides. Examples of such neurological conditions encompassed within the scope of this invention are: brain diseases, such as metabolic brain diseases which includes phenylketonuria such as maternal phenylketonuria, pyruvate carboxylase deficiency, pyruvate dehydrogenase complex deficiency, Wernicke's Encephalopathy, brain edema, brain neoplasms such as cerebellar neoplasms which include infratentorial neoplasms, cerebral ventricle neoplasms such as choroid plexus neoplasms, hypothalamic neoplasms, supratentorial neoplasms, canavan disease, cerebellar diseases such as cerebellar ataxia which include spinocerebellar degeneration such as ataxia telangiectasia, cerebellar dyssynergia, Friederich's Ataxia, Machado-Joseph Disease, olivopontocerebellar atrophy, cerebellar neoplasms such as infratentorial neoplasms, diffuse cerebral sclerosis such as encephalitis periaxialis, globoid cell leukodystrophy, metachromatic leukodystrophy and subacute sclerosing panencephalitis, carotid artery thrombosis, carotid stenosis and Moyamoya Disease), cerebral amyloid angiopathy, cerebral aneurysm, cerebral anoxia, cerebral arteriosclerosis, cerebral arteriovenous malformations, cerebral artery diseases, cerebral embolism and thrombosis such as carotid artery thrombosis, sinus thrombosis and Wallenberg's Syndrome, cerebral hemorrhage such as epidural hematoma, subdural hematoma and subarachnoid hemorrhage, cerebral infarction, cerebral ischemia such as transient cerebral ischemia, Subclavian Steal Syndrome and vertebrobasilar insufficiency, vascular dementia such as multi-infarct dementia, periventricular leukomalacia, vascular headache such as cluster headache and migraine, AIDS Dementia Complex, presenile dementia such as Alzheimer's Disease and Creutzfeldt-Jakob Syndrome, senile dementia such as Alzheimer's Disease and progressive supranuclear palsy, vascular dementia such as multi-infarct dementia, encephalitis which include encephalitis periaxialis, viral encephalitis such as epidemic encephalitis, Japanese Encephalitis, St. Louis Encephalitis, tick-borne encephalitis and West Nile Fever, acute disseminated encephalomyelitis, meningoencephalitis such as uveomeningoencephalitic syndrome, Postencephalitic Parkinson Disease and subacute sclerosing panencephalitis, encephalomalacia such as periventricular leukomalacia, epilepsy such as generalized epilepsy which includes infantile spasms, absence epilepsy, myoclonic epilepsy which includes MERRF Syndrome, tonic-clonic epilepsy, partial epilepsy such as complex partial epilepsy, frontal lobe epilepsy and temporal lobe epilepsy, post-traumatic epilepsy, status epilepticus such as Epilepsia Partialis Continua, and Hallervorden-Spatz Syndrome, hydrocephalus such as Dandy-Walker Syndrome and normal pressure hydrocephalus, hypothalamic diseases such as hypothalamic neoplasms, cerebral malaria, narcolepsy which includes cataplexy, bulbar poliomyelitis, cerebri pseudotumor, Rett Syndrome, Reye's Syndrome, thalamic diseases, cerebral toxoplasmosis, intracranial tuberculoma and Zellweger Syndrome, central nervous system infections such as AIDS Dementia Complex, Brain Abscess, subdural empyema, encephalomyelitis such as Equine Encephalomyelitis, Venezuelan Equine Encephalomyelitis, Necrotizing Hemorrhagic Encephalomyelitis, Visna, and cerebral malaria., meningitis such as arachnoiditis, aseptic meningitis such as viral meningtitis which includes lymphocytic choriomeningitis, Bacterial meningtitis which includes Haemophilus Meningtitis, Listeria Meningtitis, Meningococcal Meningtitis such as Waterhouse-Friderichsen Syndrome, Pneumococcal Meningtitis and meningeal tuberculosis, fungal meningitis such as Cryptococcal Meningtitis, subdural effusion, meningoencephalitis such as uvemeningoencephalitic syndrome, myelitis such as transverse myelitis, neurosyphilis such as tabes dorsalis, poliomyelitis which includes bulbar poliomyelitis and postpoliomyelitis syndrome.

Furthermore, other neuropathological conditions encompassed by this invention also may include: prion diseases (such as Creutzfeldt-Jakob Syndrome, Bovine Spongiform Encephalopathy, Gerstmann-Straussler Syndrome, Kuru, Scrapie), and cerebral toxoplasmosis, central nervous system neoplasms such as brain neoplasms that include cerebellar neoplasms such as infratentorial neoplasms, cerebral ventricle neoplasms such as choroid plexus neoplasms, hypothalamic neoplasms and supratentorial neoplasms, meningeal neoplasms, spinal cord neoplasms which include epidural neoplasms, demyelinating diseases such as Canavan Diseases, diffuse cerebral sceloris which includes adrenoleukodystrophy, encephalitis periaxialis, globoid cell leukodystrophy, diffuse cerebral sclerosis such as metachromatic leukodystrophy, allergic encephalomyelitis, necrotizing hemorrhagic encephalomyelitis, progressive multifocal leukoencephalopathy, multiple sclerosis, central pontine myelinolysis, transverse myelitis, neuromyelitis optica, Scrapie, Swayback, Chronic Fatigue Syndrome, Visna, High Pressure Nervous Syndrome, Meningism, spinal cord diseases such as amyotonia congenita, amyotrophic lateral sclerosis, spinal muscular atrophy such as Werdnig-Hoffmann Disease, spinal cord compression, spinal cord neoplasms such as epidural neoplasms, syringomyelia, Tabes Dorsalis, Stiff-Man Syndrome, mental retardation such as Angelman Syndrome, Cri-du-Chat Syndrome, De Lange's Syndrome, Down Syndrome, Gangliosidoses such as gangliosidoses GM1, Sandhoff Disease, Tay-Sachs Disease, Hartnup Disease, homocystinuria, Laurence-Moon-Biedl Syndrome, Lesch-Nyhan Syndrome, Maple Syrup Urine Disease, mucolipidosis such as fucosidosis, neuronal ceroid-lipofuscinosis, oculocerebrorenal syndrome, phenylketonuria such as maternal phenylketonuria, Prader-Willi Syndrome, Rett Syndrome, Rubinstein-Taybi Syndrome, Tuberous Sclerosis, WAGR Syndrome, nervous system abnormalities such as holoprosencephaly, neural tube defects such as anencephaly which includes hydrangencephaly, Arnold-Chairi Deformity, encephalocele, meningocele, meningomyelocele, spinal dysraphism such as spina bifida cystic and spina bifida occulta, Charcot-Marie Disease, Hereditary optic atrophy, Refsum's Disease, hereditary spastic paraplegia, Werdnig-Hoffmann Disease, Hereditary Sensory and Autonomic Neuropathies such as Congenital Analgesia and Familial Dysautonomia, Neurologic manifestations (such as agnosia that include Gerstmann's Syndrome, Amnesia such as retrograde amnesia, apraxia, neurogenic bladder, and cataplexy.

Also encompassed within the scope of this invention are communicative disorders such as hearing disorders that include deafness, partial hearing loss, loudness recruitment and tinnitus, language disorders such as aphasia which include agraphia, anomia, broca aphasia, and Wernicke Aphasia, Dyslexia such as Acquired Dyslexia, language development disorders, speech disorders such as aphasia which includes anomia, broca aphasia and Wernicke Aphasia, articulation disorders, communicative disorders such as speech disorders which include dysarthria, echolalia, mutism and stuttering, voice disorders such as aphonia and hoarseness, decerebrate state, delirium, fasciculation, hallucinations, meningism, movement disorders such as angelman syndrome, ataxia, athetosis, chorea, dystonia, hypokinesia, muscle hypotonia, myoclonus, tic, torticollis and tremor, muscle hypertonia such as muscle rigidity such as stiff-man syndrome, muscle spasticity, paralysis such as facial paralysis which includes Herpes Zoster Oticus, Gastroparesis, Hemiplegia, ophthalmoplegia such as diplopia, Duane's Syndrome, Homer's Syndrome, Chronic progressive external ophthalmoplegia such as Kearns Syndrome, Bulbar Paralysis, Tropical Spastic Paraparesis, and Paraplegia such as Brown-Sequard Syndrome.

The following neurological conditions are also encompassed within the scope of this invention: quadriplegia, respiratory paralysis and vocal cord paralysis, paresis, phantom limb, taste disorders such as ageusia and dysgeusia, vision disorders such as amblyopia, blindness, color vision defects, diplopia, hemianopsia, scotoma and subnormal vision, sleep disorders such as hypersomnia which includes Kleine-Levin Syndrome, insomnia, and somnambulism, spasm such as trismus, unconsciousness such as coma, persistent vegetative state and syncope and vertigo, neuromuscular diseases such as amyotonia congenita, amyotrophic lateral sclerosis, Lambert-Eaton Myasthenic Syndrome, motor neuron disease, muscular atrophy such as spinal muscular atrophy, Charcot-Marie Disease and Werdnig-Hoffmann Disease, Postpoliomyelitis Syndrome, Muscular Dystrophy, Myasthenia Gravis, Myotonia Atrophica, Myotonia Confenita, Nemaline Myopathy, Familial Periodic Paralysis, Multiplex Paramyloclonus, Tropical Spastic Paraparesis and Stiff-Man Syndrome, peripheral nervous system diseases such as acrodynia, amyloid neuropathies, autonomic nervous system diseases such as Adie's Syndrome, Barre-Lieou Syndrome, Familial Dysautonomia, Homer's Syndrome, Reflex Sympathetic Dystrophy and Shy-Drager Syndrome, Cranial Nerve Diseases such as Acoustic Nerve Diseases such as Acoustic Neuroma which includes Neurofibromatosis 2, Facial Nerve Diseases such as Facial Neuralgia,Melkersson-Rosenthal Syndrome, ocular motility disorders which includes amblyopia, nystagmus, oculomotor nerve paralysis, ophthalmoplegia such as Duane's Syndrome, Homer's Syndrome, Chronic Progressive External Ophthahnoplegia which includes Kearns Syndrome, Strabismus such as Esotropia and Exotropia, Oculomotor Nerve Paralysis, Optic Nerve Diseases such as Optic Atrophy which includes Hereditary Optic Atrophy, Optic Disk Drusen, Optic Neuritis such as Neuromyelitis Optica, Papilledema, Trigeminal Neuralgia, Vocal Cord Paralysis, Demyelinating Diseases such as Neuromyelitis Optica and Swayback, and Diabetic neuropathies such as diabetic foot, nerve compression syndromes such as carpal tunnel syndrome, tarsal tunnel syndrome, thoracic outlet syndrome such as cervical rib syndrome, ulnar nerve compression syndrome, neuralgia such as causalgia, cervico-brachial neuralgia, facial neuralgia and trigeminal neuralgia, neuritis such as experimental allergic neuritis, optic neuritis, polyneuritis, polyradiculoneuritis and radiculities such as polyradiculitis, hereditary motor and sensory neuropathies such as Charcot-Marie Disease, Hereditary Optic Atrophy, Refsum's Disease, Hereditary Spastic Paraplegia and Werdnig-Hoffmann Disease, Hereditary Sensory and Autonomic Neuropathies which include Congenital Analgesia and Familial Dysautonomia, POEMS Syndrome, Sciatica, Gustatory Sweating and Tetany.

All of the above-identified neurological disorders and their associated gastrointestinal problems may be ameliorated or prevented by the administration of enhancer molecules, such as those discussed hereinabove. These enhancer molecules are useful as active compounds in pharmaceutical compositions. The pharmacologically active compositions of this invention can be processed in accordance with conventional methods of pharmacy to produce pharmaceutical agents and/or dietary supplements for administration to patients, e.g., in admixtures with conventional excipients such as pharmaceutically acceptable organic or inorganic carrier substances suitable for parenteral, enteral (e.g., oral), topical or transdermal application which do not deleteriously react with the active compounds. Suitable pharmaceutically acceptable carriers include but are not limited to water, salt (buffer) solutions, alcohols, gum arabic, mineral and vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc.

A probiotic preparation or supplementation which reduces lactic acid accumulation by: formation of alternative end products of fermentation; production of volatile fatty acids rather than lactic acid during carbohydrate fermentation; through increased utilization of lactic acid; or through the conversion of lactic acid to volatile fatty acids which can be absorbed from the gut, thereby reducing acidity in the gut. Probiotic preparations may include bacteria which ferment starch and sugars to end products other than lactic acid, (i.e., volatile fatty acids). Probiotic preparations may include bacteria selected from the group consisting of: Succinomonas, Butyrivibrio, Bacteroides and Succinivibrio. These bacteria can be used individually or in combination. Typically, preferred probiotic preparations include bacteria capable of utilizing lactic acid, and converting lactic acid to volatile fatty acids and other end products, anaerobic bacteria, or bacteria selected from the group consisting of: Megasphera, Veillenolla, Selenomonas, Propionibacterium, Anaerovibrio and Peptococcus. These bacteria can be used individually or in combination. Also, probiotic preparations may include yeast and mycelial preparations capable of utilizing lactic acid, and converting lactic acid to volatile fatty acids and other end products.

The pharmaceutical preparations can be sterilized and if desired, mixed with auxiliary agents, e.g., lubricants, preservatives, stabilizers, wetting agents, emulsifiers, salts for influencing osmotic pressure, buffers, coloring, flavoring and/or aromatic active compounds. If a pharmaceutically acceptable solid carrier is used, the dosage form of the analogs may be tablets, capsules, powders, suppositories, or lozenges. If a liquid carrier is used, soft gelatin capsules, transdermal patches, aerosol sprays, topical creams, syrups or liquid suspensions, emulsions or solutions may be the dosage form.

For parenteral application, particularly suitable are injectable, sterile solutions, preferably oily or aqueous solutions, as well as suspensions, emulsions, or implants, including suppositories. Ampoules are convenient unit dosages. The dosage of the pharmaceutically active enhancer compositions for parenteral administration generally is about 10-100 milligrams given 1 to 5 times per week.

As noted above, for enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., liposomes or those wherein the active compound is protected with differentially degradable coatings, such as by microencapsulation, multiple coatings, etc. It is also possible to freeze-dry the new compounds and use the lypolizates obtained, for example, for the preparation of products for injection. Transdermal delivery of the pharmaceutically active enhancer compositions is also possible.

A sustained release formulation which releases the active ingredient over a period of time (preferably in the ileum or colon) and thereby reduces the number of daily doses is advantageous. Such a sustained release formulation may use any conventional sustained release component such as (for example) a barrier, coating or erodable matrix.

For topical application, there are employed as nonsprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, etc.

Oral administration is preferred. Generally, the pharmaceutically active enhancer compositions of this invention are dispensed by unit dosage form comprising about 5 to about 50 milligrams in a pharmaceutically acceptable carrier per unit dosage. The dosage of the enhancer compositions generally is about 100 to about 500 milligrams per week depending on the weight of the subject and the severity of the neurological disorder and the associated gastrointestinal problems. This is readily determined by a person skilled in the art for the chosen polyglycoside.

Typically the daily dose for a child under three years would be 50 mg orally and for a child between three and five years would be 100 mg orally. For a subject from the age of 6 years upwards the dose would typically be 100 mg twice daily. If adverse affects occur such as increased diarrhea results, the dose should be reduced.

Typically the selected dose is administered three times daily (e.g., orally) and the blood should be monitored to ensure that large amounts are not being absorbed after 48 hours. The administration regime will typically continue for several months (e.g., three to six months or longer) before any long term benefit is observed.

It will be appreciated that the actual preferred amounts of pharmaceutically active enhancer compositions in a specific case will vary according to the specific compound being utilized, the particular compositions formulated, the mode of application, and the particular situs being treated. Dosages can be determined using conventional considerations, e.g., by customary comparison of the differential activities of the subject compounds and of a known agent, e.g., by means of an appropriate conventional pharmacological protocol.

The specific doses for each particular patient depends on a wide variety of factors, for example, on the efficacy of the specific compound employed, the particular compositions formulated, the mode of application, and the particular situs and organism being treated. For example, the specific dose for a particular patient depends on age, sex, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

The dosage forms may also contain adjuvants, such as preserving or stabilizing adjuvants. They may also contain other therapeutically valuable substances or may contain more than one of the compounds specified herein and in the claims in admixture.

As described hereinbefore, the pharmaceutically active enhancer compositions may be administered to mammals, but preferably administered to the human patients in oral dosage formulation so that it can be absorbed from the intestine into the blood.

Although the above description provides for administration by mouth, it is to be understood that the compounds can also be administered in alternative fashions, including intranasally, transdermally, intrarectally, intravaginally, subcutaneously, intravenously, and intramuscularly.

The examples are being described for purely illustrative purposes, and are in no way meant to limit the scope of the invention.

While the above example was directed to the treatment of CHDRF, the same or a similar administration may be used for the treatment of the Syndrome of Lipodystrophy or one or more of the HIV-related abnormalities included therein.

### EXAMPLE

### Example 1

### Treatment of a Neurological Disease Associated With Gastrointestinal Conditions

A patient with the symptoms of a neurological disease associated with gastrointestinal conditions is administered a treatment regimen of the composition as defined herein which includes at least one of a peptide, a metal, a saccharide, and a probiotic, or a combination thereof.

The patient in the present example developed normally until about fourteen months of age, with the exception of gastrointestinal problems (i.e., chronic diarrhea and constipation) which began at about six months. At about thirteen months, when whole milk was introduced into his diet, the patient began having reoccurring ear infections. At about fourteen months, the patient appeared to lose the ability to process language, first receptively (at about 14 months) then expressively (at about 16 months). The patient also experienced episodes of “shivers” that appeared to be intermittent seizures.

The dose of 250 milligrams per week of the composition in accordance with the present invention is administered in the subject. The dose indicated herein is for reference purposes and may vary between 100 to 500 milligrams per week, depending on the characteristics of the patient. Amounts may be generally proportional to the amounts set forth herein, relative to the strength and potency of the particular composition administered.

After a treatment period of weeks to months, the patient typically would show improved digestion of diet derived biomolecules such as casein and gluten, resulting in a reduction of peptides with opioid type properties in the urine of patients, including reduced uptake of neuroactive diet-derived opioid compounds from the gut by restoring normal gut permeability.

Furthermore, diagnostic testing reveals that the patient's pancreas would respond, with an unusually large amount of pancreatic juice being released (approximately 10 ml/min compared to a usual rate of 1-2 ml/min). The diagnostic tests to be performed on the patient would also indicate gastric inflammation. Within days after the administration of the composition suggested herein, the patient's chronic abnormal bowel movement becomes normal, although no changes would be made in the patient's diet. Within weeks after the treatment, the patient demonstrates signs of improvement in both behavior and development. Biomedical changes are also measured in the subject. A single photon emission computed tomography, SPECT scan of the patient indicates that the perfusion of the right posterior parietal and right temporal lobes was improved. Blood tests taken after the treatment also indicates a rise in serotonin levels, and a drop in the subject's rubella titers to a more normal rate. Thus, the results demonstrate the oral administration of the composition improves neurological diseases and their related gastrointestinal conditions.

In summary, the present invention provides a therapeutic composition and method for enhancing expression of genes and enhancing the activity of enzymes, which are present in the gastrointestinal tract and wherein their reduced activity has been linked to numerous neurological disorders.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation that lawfully can be accorded the appended claims.

In addition, all publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A composition for the treatment of a neurological disorder and an associated gastrointestinal condition, the composition comprising a therapeutically effective amount of at least one of a peptide, a saccharide, a probiotic, a metal and a combinations thereof, the composition having the ability to enhance expression of genes, enhance enzymatic activity of gastrointestinal proteins, or both.

2. A composition as set forth in claim 1, wherein the composition comprises the probiotic.

3. A composition as set forth in claim 2, wherein the probiotic is bacterial in origin.

4. A composition as set forth in claim 2, wherein the probiotic includes at least one of Lactobacillus acidophilus and Bifidobacterium bifidum.

5. A composition as set forth in claim 2, wherein the probiotic includes at least one of: Succinomonas, Butyrivibrio, Bacteroides, Succinivibrio, and a combination thereof.

6. A composition as set forth in claim 2, wherein the probiotic includes at least one of: Megasphera, Veillenolla, Selenomonas, Propionibacterium, Anaerovibrio, Peptococcus, and a combination thereof.

7. A composition as set forth in claim 1, wherein the probiotic is yeast or mycelial in origin.

8. A composition as set forth in claim 7, wherein the probiotic is capable of utilizing lactic acid, and converting lactic acid to volatile fatty acids and other end products.

9. A composition as set forth in claim 1, wherein the composition comprises the peptide.

10. A composition as set forth in claim 9, wherein the peptide ranges in length from 1-30 amino acids.

11. A composition as set forth in claim 9, wherein the peptide includes at least one of: a mono-peptide, di-peptide, tri-peptide and a conservative derivative thereof.

12. A composition as set forth in claim 11, wherein the mono-peptide includes at least one of: Alanine, Arginine, Glycine, Glutamine, Glutamic Acid, Isoleucine, Phenylalanine, Serine, Threonine, Valine, and a conservative derivative thereof.

13. A composition as set forth in claim 11, wherein the di-peptide includes at least one of: Gly-Ala, Gly-Gly, Gly-Ile, Gly-D-Ala, Gly-Arg, Gly-Asp, Gly-Asn, Gly-Gln, Gly-Glu, Gly-Leu, Gly-Phe, Gly-Pro, Gly-Ser, Gly-Thr, and a conservative derivative thereof.

14. A composition as set forth in claim 11, wherein the tri-peptide includes at least one of: Gly-Gly-Gly and a conservative derivative thereof.

15. A composition as set forth in claim 1, wherein the composition comprises the saccharide.

16. A composition as set forth in claim 15, wherein the saccharide is a mono- or poly-saccharide.

17. A composition as set forth in claim 16, wherein the mono-saccharide includes at least one of: pentose, ribose, hexose, glucose, fructose, mannose, glucosamine, galactose, galactosamine, xylose, and a monoglycoside sugar analog thereof.

18. A composition as set forth in claim 16, wherein the poly-saccharide includes at least one of: lactose, sucrose, iduronic acid, glycimic acid, starches, dextrin, oligoglycoside or a naturally occurring and synthetic sugar derivative.

19. A composition as set forth in claim 1, wherein the composition comprises the metal.

20. A composition as set forth in claim 19, wherein the metal is capable of interacting with a protein active site(s) so as to enhance enzyme activity.

21. A composition as set forth in claim 20, wherein the protein exhibits reduced enzymatic activity.

22. A composition as set forth in claim 19, wherein the metal includes at least one of: iron, iron-sulfur, copper, manganese, selenium, zinc, aluminum, and combinations thereof.

23. A composition as set forth in claim 1, wherein the neurological disorder includes at least one of autism, autism spectrum disorders; Parkinson's disease; cognitive impairments; age-associated memory impairments; cognitive impairments; dementia associated with neurologic and/or psychiatric conditions; epilepsy; brain tumors; brain lesions; multiple sclerosis; Down's syndrome; Rett's syndrome; progressive supranuclear palsy; frontal lobe syndrome; schizophrenia and related psychiatric disorders; delirium; Tourette's syndrome; myasthenia gravis; attention deficit hyperactivity disorder; dyslexia; mania; depression; apathy; myopathy; Alzheimer's disease; Huntington's Disease; dementia; schizophrenia; severe clinical depression; brain injury; ADD (Attention Deficit Disorder); ADHD (Attention Deficit Hyperactivity Disorder); hyperactivity disorder; Asperger's Disorder; and other pervasive developmental disorders.

24. A composition as set forth in claim 23, wherein the neurological disorder autism of the type affecting activity of DPPIV.

25. A composition as set forth in claim 1, wherein the gastrointestinal condition includes at least one of nausea, abdominal pain, constipation, diarrhea, Functional Bowel Disorder, Irritable Bowel Syndrome, Crohn's disease, ulcers, heartburn, irregularity, gastric neurosis, diverticulosis, cirrhosis, celiac disease, acute gastritis, dyspepsia, gastralgia, gastric carcinoma, gastric vertigo, enteritis, peptic ulcers, cholera morbus, cholera infantum, gastroenteritis, bloating, flatulence, inflammatory bowel disease, acid reflux disease, abdominal pain, and Ulcerative colitis.

26. A method for treating a neurological disorder and an associated gastrointestinal condition, the method comprising administering a therapeutically effective amount of a composition comprising at least one of a peptide, a saccharide, a probiotic, a metal and a combination thereof, the method having the ability to enhance expression of genes, enhance enzymatic activity of gastrointestinal proteins, or both.

27. A method as set forth in claim 26, wherein the composition comprises the probiotic.

28. A method as set forth in claim 27, wherein the probiotic is bacterial in origin.

29. A method as set forth in claim 27, wherein the probiotic includes at least one of Lactobacillus acidophilus and Bifidobacterium bifidum.

30. A method as set forth in claim 27, wherein the probiotic includes at least one of: Succinomonas, Butyrivibrio, Bacteroides, Succinivibrio, and a combination thereof.

31. A method as set forth in claim 27, wherein the probiotic includes at least one of: Megasphera, Veillenolla, Selenomonas, Propionibacterium, Anaerovibrio, Peptococcus, and a combination thereof.

32. A method as set forth in claim 27, wherein the probiotic is yeast or mycelial in origin.

33. A method as set forth in claim 32, wherein the probiotic is capable of utilizing lactic acid, and converting lactic acid to volatile fatty acids and other end products.

34. A method as set forth in claim 26, wherein the composition comprises the peptide.

35. A method as set forth in claim 34, wherein the peptide ranges in length from 1-30 amino acids.

36. A method as set forth in claim 34, wherein the peptide includes at least one of: a mono-peptide, di-peptide, tri-peptide, and a conservative derivatives thereof.

37. A method as set forth in claim 36, wherein the mono-peptide includes at least one of: Alanine, Arginine, Glycine, Glutamine, Glutamic Acid, Isoleucine, Phenylalanine, Serine, Threonine, Valine, and a conservative derivative thereof.

38. A method as set forth in claim 36, wherein the di-peptide includes at least one of: Gly-Ala, Gly-Gly, Gly-Ile, Gly-D-Ala, Gly-Arg, Gly-Asp, Gly-Asn, Gly-Gln, Gly-Glu, Gly-Leu, Gly-Phe, Gly-Pro, Gly-Ser, Gly-Thr, and a conservative derivatives thereof.

39. A method as set forth in claim 36, wherein the tri-peptide includes at least one of: Gly-Gly-Gly and a conservative derivative thereof.

40. A method as set forth in claim 26, wherein the composition comprises the saccharide.

41. A method as set forth in claim 40, wherein the saccharide is a mono- or polysaccharide.

42. A method as set forth in claim 40, wherein the mono-saccharide includes at least one of: pentose, ribose, hexose, glucose, fructose, mannose, glucosamine, galactose, galactosamine, xylose, and a monoglycoside sugar analog.

43. A method as set forth in claim 40, wherein the poly-saccharide includes at least one of: lactose, sucrose, iduronic acid, glycimic acid, starches, dextrins oligoglycoside and other a naturally occurring and synthetic sugar derivative.

44. A method as set forth in claim 26, wherein the composition comprises the metal.

45. A method as set forth in claim 44, wherein the metal is capable of interacting with a protein active site(s) so as to enhance the enzyme activity.

46. A method as set forth in claim 45, wherein the protein exhibits reduced enzymatic activity.

47. A method as set forth in claim 44, wherein the metal includes at least one of: iron, iron-sulfur, copper, manganese, selenium, zinc, aluminum, and combinations thereof.

48. A method as set forth in claim 26, wherein the neurological disorder includes at least one of autism, autism spectrum disorders; Parkinson's disease; cognitive impairments; age-associated memory impairments; cognitive impairments; dementia associated with neurologic and/or psychiatric conditions; epilepsy; brain tumors; brain lesions; multiple sclerosis; Down's syndrome; Rett's syndrome; progressive supranuclear palsy; frontal lobe syndrome; schizophrenia and related psychiatric disorders; delirium; Tourette's syndrome; myasthenia gravis; attention deficit hyperactivity disorder; dyslexia; mania; depression; apathy; myopathy; Alzheimer's disease; Huntington's Disease; dementia; schizophrenia; severe clinical depression; brain injury; ADD (Attention Deficit Disorder); ADHD (Attention Deficit Hyperactivity Disorder); hyperactivity disorder; Asperger's Disorder; and other pervasive developmental disorders.

49. A method as set forth in claim 48, wherein the neurological disorder autism of the type affecting activity of DPPIV.

50. A method as set forth in claim 26, wherein the gastrointestinal condition includes at least one of nausea, abdominal pain, constipation, diarrhea, Functional Bowel Disorder, Irritable Bowel Syndrome, Crohn's disease, ulcers, heartburn, irregularity, gastric neurosis, diverticulosis, cirrhosis, celiac disease, acute gastritis, dyspepsia, gastralgia, gastric carcinoma, gastric vertigo, enteritis, peptic ulcers, cholera morbus, cholera infantum, gastroenteritis, bloating, flatulence, inflammatory bowel disease, acid reflux disease, abdominal pain, and Ulcerative colitis.
